(19) United States
(12) Patent Application Publication
(10) Pub. No.: US 2026/0345573 A1
(43) Pub. Date: Nov. 5, 2026

(54) METHOD OF TREATMENT WITH ILOPERIDONE

(71) Applicant: Vanda Pharmaceuticals Inc., Washington, DC (US)

(72) Inventors: Mihael H. Polymeropoulos, Potomac, MD (US); Sandra Smieszek, Washington, DC (US)

(21) Appl. No.: 19/487,752

(22) PCT Filed: May 31, 2024
(86) PCT No.: PCT/US2024/032137

§ 371 Date: Nov. 25, 2025

Related U.S. Application Data
(60) Provisional application No. 63/505,884, filed on Jun. 2, 2023.

(51) Int. Cl. A61K 31/454; A61K 9/00; A61P 19/06; G01N 33/62

(52) U.S. Cl. CPC A61K 31/454; A61K 9/0053; A61P 19/06; G01N 33/62; G01N 2800/107; G01N 2800/52

(57) ABSTRACT

Described herein is an improved method of treatment with iloperidone or an active metabolite thereof, of a patient in need of such treatment, where such patient has a history of gout or is a carrier of a genetic variant associated with iloperidone-induced increases in serum urate concentration. The improved method comprises monitoring a serum urate level of the patient; and in an event that the serum urate level of the patient exceeds a reference level, initiating treatment with, or increasing a dose of a urate lowering treatment.

### CROSS REFERENCE TO RELATED APPLICATION

This patent application claims priority to U.S. Provisional Patent Application No. 63/505,884, filed Jun. 2, 2023.

### BACKGROUND OF THE INVENTION

The invention relates generally to improvements in methods of treatment with iloperidone. More particularly, the invention relates to improved methods of treatment with iloperidone which limit risk of hyperuricemia and gout.

### Iloperidone

Iloperidone (1-[4-[3-[4-(6-flouro-1,2-benzisoxazol-3-yl)-1-piperidinyl]propoxy]-3-methoxyphenyl]ethanone) is an atypical antipsychotic disclosed in U.S. Pat. RE39198 (Jul. 18, 2006). Iloperidone is approved for use in the United States for the treatment of schizophrenia in adults and for the acute treatment of manic or mixed episodes associated with bipolar I disorder in adults. Iloperidone is also in development for use in the treatment of other psychotic symptoms, diseases, and disorders.

1-[4-[3-[4-(6-fluoro-1,2-benzisoxazol-3-yl)-1-piperidinyl]propoxy]-3-methoxyphenyl]ethanol), also known by the chemical name 4-[3-[4-(6-fluoro-1,2-benzisoxazol-3-yl)-1-piperidinyl]propoxy]-3-methoxy-α-methylbenzenemethanol, and as(S)-P-88-8891, S-P88, or P88, is an active metabolite of iloperidone. In humans, P88 is only found in the S-enantiomeric form, which has the following structure:

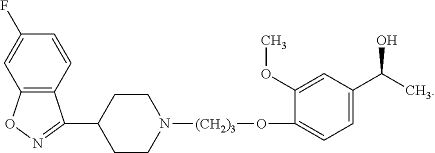

However, P88 can also be synthesized in its R-enantiomeric form, which has the structure:

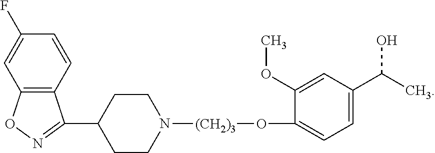

P88, and the S- and R-enantiomeric forms thereof, are described in U.S. Pat. No. 7,977,356 (Jul. 12, 2011) and 10,874,659 (Dec. 29, 2020), respectively.

Other iloperidone metabolites include: 1-[4-[3-[4-(6-fluoro-1,2-benzisoxazol-3-yl)-1-piperidinyl]propoxy]-3-hydroxyphenyl]ethanone; 1-[4-[3-[4-(6-fluoro-1,2-benzisoxazol-3-yl)-1-piperidinyl]propoxy]-3-methoxyphenyl]-2-hydroxyethanone; 4-[3-[4-(6-fluoro-1,2-benzisoxazol-3-yl)-1-piperidinyl]propoxy]-3-hydroxy-α-methylbenzene methanol; 4-[3-[4-(6-fluoro-1,2-benzisoxazol-3-yl)-1-piperidinyl]propoxyl-2-hydroxy-5-methoxy-α-methylbenzenemethanol; 1-[4-[3-[4-(6-fluoro-1,2-benzisoxazol-3-yl)-1-piperidinyl]propoxy]-2-hydroxy-5-methoxyphenyl]ethanone; and 1-[4-[3-[4-(6-fluoro-1,2-benzisoxazol-3-yl)-1-piperidinyl]propoxy]-2,5-dihydroxyphenyl]ethanone. See, e.g., U.S. Pat. No. 5,364,866 (Nov. 15, 1994), and International Patent Application Publication No. WO 95/11680 A1 (May 4, 1995).

Previous studies have investigated associations between iloperidone efficacy and polymorphisms in genes and gene regions including CFTR, NPAS3, XKR4, TNR, GRIA4, GFRA2, and NUDT9P1. These associations are described in, e.g., U.S. Pat. No. 9,328,387 (May 3, 2016), U.S. Pat. No. 9,458,507 (Oct. 4, 2016), and U.S. Pat. No. 9,080,214 (Jul. 14, 2015). Additionally, associations between CYP2D6 genotypes and changes in QT interval following the administration of iloperidone are described in U.S. Pat. No. 8,586,610 (Nov. 19, 2013) and U.S. Pat. No. 9,138,432 (Sep. 22, 2015). Associations between KCNQ1 genotypes and iloperidone administration-induced changes in QT interval are described in U.S. Pat. No. 8,999,638 (Apr. 7, 2015), and U.S. Pat. No. 9,157,121 (Oct. 13, 2015). Such findings relating to the safety and efficacy of iloperidone aid in selecting the most optimal drug and dosage regimen for a particular patient. This in turn aids in safe and effective treatment of psychotic symptoms, diseases, and disorders, with less trial and error, and a reduction in deleterious side effects.

### Serum Urate Concentration (SUC)

Uric acid is the end product of purine metabolism in humans and primates, which lack the enzyme uricase to further oxidize the heterocyclic compound. Under normal conditions the urate anion is generated after breakdown of excess purine nucleosides in the liver and the majority of the molecule is then excreted through the kidneys via urine. Hyperuricemia, or elevated levels of serum urate (uric acid in the blood), is a significant risk factor for gout, a common and complex inflammatory arthritis characterized by painful recurrent attacks of swelling and tenderness of the joints. Excess urate in the body causes gout due to deposition of monosodium urate crystals (i.e. tophi) in cartilage tissues, tendons, and soft tissues. Crystal deposits can also form in the kidneys, leading to uric acid kidney stones and in chronic cases, renal failure.

The concentration of serum urate in individuals is influenced by several factors, most notably genetic polymorphisms of renal transporters, medications, and diet. Levels of serum urate correlate with blood pressure, metabolic syndrome, diabetes, gout, and cardiovascular disease. Commercial laboratory tests are routinely used to evaluate the presence of uric acid in the blood because imbalance in serum urate is an established marker of underlying disease states, and facilitates diagnosis of gout, renal or kidney impairment, and rare inherited conditions.

Bidirectional transport of urate and the presence of hepatic uricase in non-primate mammalian models limited the early study of uric acid transport in humans. However, genetic methods have facilitated the discovery of transport proteins as well as the functional impact of genetic polymorphisms in the genes that encode them. The SLC2A9 gene encodes a high-capacity hexose-urate transporter in humans and is mainly expressed in kidneys, liver, and intestines. Loss of function variants of SLC2A9 were previously identified in association with hypouricemia, and recent genome-wide association studies have since correlated common genetic variants of SLC2A9 with increased concentrations of serum urate and gout.

### BRIEF DESCRIPTION OF THE INVENTION

Various aspects of the invention disclosed herein related to improved methods for the treatment of a patient in need thereof, with iloperidone, while reducing risk of hyperuricemia and gout.

In a first aspect, an improved method is provided for the treatment of a patient in need thereof with iloperidone, an active metabolite of iloperidone such as P88, or a pharmaceutically acceptable salt of iloperidone or the metabolite thereof, wherein the patient has a history of gout. In such improved method, the improvement comprises: monitoring a serum urate level of the patient; and in an event that the serum urate level of the patient approaches or exceeds a reference level, initiating treatment with, or increasing a dose of a urate lowering treatment.

In a second aspect an improved method is provided for the treatment of a patient in need thereof with iloperidone, an active metabolite of iloperidone such as P88, or a pharmaceutically acceptable salt of iloperidone or the metabolite thereof, wherein the patient has a genotype associated with iloperidone-induced increases in serum urate concentration such as, e.g., a rs7442295 genotype at the SLC2A9 gene locus that includes one or more rs7442295 (G) alleles. In such improved method, the improvement comprises: monitoring a serum urate level of the patient; and in an event that the serum urate level of the patient approaches or exceeds a reference level, initiating treatment with, or increasing a dose of a urate lowering treatment.

These and other aspects, advantages, and salient features of the invention will become apparent from the following detailed description, which, when taken in conjunction with the annexed drawings, disclose embodiments of the invention.

### BRIEF DESCRIPTION OF THE DRAWINGS

FIG. 1 plots the mean change from baseline to day 28 for serum urate in observed cases over time in the study described in Example 1 (bipolar mania) and the study described in Example 2 (schizophrenia). In the figure, error bars represent standard error of the mean (SEM).

FIG. 2 plots baseline evaluations of serum urate according to patient genotype and sex in the study described in Example 1.

FIG. 3 plots individual patient changes from baseline to Day 28 grouped according to treatment group and carrier status for variant rs7442295 in the study described in Example 1, in which the red line represents mean.

FIG. 4 plots the mean concentrations of serum urate at baseline and Day 28 by genotype and treatment for observed cases in the study described in Example 1, in which error bars represent the standard error of the mean.

FIG. 5 plots the change from baseline in serum urate by sex in the study described in Example 1.

FIGS. 6A (females) and 6B (males) illustrate the percentage of individuals, stratified by sex, and their shifts in serum urate concentration. These figures illustrate the clinical relevance of the rs7442295-G/G genotype, in which a significant number of individuals (particularly homozygous G/G males) exceed the upper limit of normal (ULN) when treated with iloperidone as compared to placebo.

FIG. 7 plots change from baseline to end of study (EOS) in serum urate by treatment group and carrier status for rs7442295 in the study described in Example 2, in which red line represents mean, and red error bars represent standard error of the mean (SEM).

FIG. 8 plots mean serum urate at baseline and EOS by carrier status of variant rs7442295 in the study described in Example 2, in which error bars represent standard error of the mean.

FIG. 9 plots change from baseline in serum urate by sex in the study described in Example 2.

The drawings are intended to depict only typical aspects of the disclosure, and therefore should not be considered as limiting the scope of the disclosure.

### DETAILED DESCRIPTION OF THE INVENTION

In various embodiments of the invention, improved methods are described herein for treatment of patients with iloperidone, active metabolites of iloperidone, or pharmaceutically acceptable salts of iloperidone or active metabolites thereof. Such active metabolites may include, e.g., P88. Such improved methods are useful to safely and effectively treat a patient in need of treatment with iloperidone, while limiting, mitigating, reducing, or avoiding the risk of certain deleterious adverse events associated with iloperidone treatment such as, e.g., hyperuricemia and gout.

As used herein, the terms “patient,” “subject,” and “individual” refer to a mammal that is afflicted with one or more disorders ameliorated by administration of iloperidone such as, e.g., schizophrenia, a schizophreniform disorder, bipolar I disorder, acute manic and mixed episodes associated with bipolar I disorder, agitation associated with Alzheimer's Disease, agitation associated with dementia, agitation associated with autism, Parkinson's Disease Psychosis, or another psychotic disease or disorder. Guinea pigs, dogs, cats, rats, mice, horses, cattle, sheep, and humans are examples of mammals within the scope of the meaning of the term. It will be understood that the most preferred patient is a human. A “patient in need of treatment with iloperidone” refers to a patient suffering from, or diagnosed with a condition that is understood by a person of skill in the art, to be treated or treatable with iloperidone, an active metabolite of iloperidone such as P88, or a pharmaceutically acceptable salt of iloperidone or the metabolite thereof, such as (but not limited to) those conditions listed above.

It is also recognized that one skilled in the art may affect the disorders discussed herein by treating a patient presently afflicted with the disorders or by prophylactically treating a patient afflicted with the disorders with an effective amount of iloperidone, an active metabolite of iloperidone such as P88, or a pharmaceutically acceptable salt of iloperidone or the metabolite thereof. Thus, the terms “treatment” and “treating” are intended to refer to all processes wherein there may be a slowing, interrupting, arresting, controlling, or stopping of the progression of the diseases or disorders described herein, or a reduction in the frequency of episodes thereof, and is intended to include prophylactic treatment of such disorders. “Treatment” does not necessarily indicate a total elimination of all disorder symptoms, and may include improvement therein.

As used herein, the term “effective amount” as it relates to iloperidone, an active metabolite of iloperidone such as P88, or a pharmaceutically acceptable salt of iloperidone or the metabolite thereof, refers to an amount of the compound that is effective in treating the disorders described herein. Non-limiting exemplary effective amounts, or effective doses, of iloperidone include titrated doses of 4-24 mg/day, 8-24 mg/day, 4-16 mg/day, 12-14 mg/day, 12-16 mg/day, 12-24 mg/day, 16-24 mg/day, 20-24 mg/day, 12 mg/day or less, or up to about 24 mg/day, e.g., 6 mg/day, 8 mg/day, 12 mg/day, 14 mg/day, 16 mg/day, 20 mg/day, 24 mg/day, 8-24 mg/day given as 4-12 mg twice daily (bid), 12-16 mg/day given as 6-8 mg bid, 12-24 mg/day given as 6-12 mg bid, 16-24 mg/day given as 8-12 mg bid, 12 mg or less per day given as 6 mg or less bid, 16 mg or less per day given as 8 mg or less bid, 16 mg/day given as 8 mg bid, 24 mg/day given as 12 mg bid, or 24 mg/day given as 24 mg once daily (qd), as disclosed in, e.g., U.S. Pat. No. 8,586,610 (Nov. 19, 2013); U.S. Pat. No. 9,138,432 (Sep. 22, 2015); U.S. Pat. No. 10,272,076 (Apr. 30, 2019); U.S. Pat. No. 10,441,580 (Oct. 15, 2019); and 10,987,346 (Apr. 27, 2021). Exemplary effective amounts, or effective doses, of R-P88 include titrated doses of 1-24 mg/day, 6-24 mg/day, 12-24 mg/day, 6-18 mg/day, or 6-12 mg/day, or up to 24 mg/day, e.g., 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 14 mg/day, 16 mg/day, 18 mg/day, 20 mg/day, 22 mg/day, or 24 mg/day; 1-24 mg/day given as 0.5-12 mg bid, 2-18 mg/day given as 1-9 mg bid, 6-18 mg/day given as 3-9 mg bid, 6-12 mg/day given as 3-6 mg bid, 6-18 mg/day, given as 3-9 mg bid; 12 mg/day given as 6 mg bid, 24 mg/day given as 12 mg bid, or up to 24 mg/day given as up to 12 mg bid, as disclosed in, e.g., U.S. Pat. No. 10,874,659 (Dec. 29, 2020).

With regard to dosing, qd refers to dosing once per day; and bid dosing typically means dosing once in the morning and once in the evening, generally no less than about 8 hours or more than about 16 hours apart, e.g., 10 to 14 hours apart, or 12 hours apart (Q12H).

As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by the context (e.g., includes the degree of error associated with measurement of the particular quantity). The suffix “(s)” as used herein is intended to include both the singular and the plural of the term that it modifies, thereby including one or more of that term (e.g., the milestone(s) includes one or more milestones). Ranges disclosed herein are inclusive and independently combinable (e.g., ranges of “up to about 25 mg, or, more specifically, about 5 mg to about 20 mg,” is inclusive of the endpoints and all intermediate values of the ranges of “about 5 mg to about 25 mg,” etc.).

In one embodiment of the invention, an improvement is provided in a method of treatment of a patient with a compound that is iloperidone, an active metabolite of iloperidone such as P88, or a pharmaceutically acceptable salt of iloperidone or the metabolite thereof. The patient may be in need of treatment with iloperidone or the salt or metabolite thereof for control of symptoms and/or prevention of relapse of, e.g., schizophrenia, a schizophreniform disorder, bipolar I disorder, bipolar mania, acute manic and mixed episodes associated with bipolar I disorder, agitation associated with Alzheimer's Disease, agitation associated with dementia, agitation associated with autism, Parkinson's Disease Psychosis, and other psychotic diseases and disorders as is known and understood in the art. In certain embodiments, the patient's treatment may include administration of iloperidone at a dose of up to 24 mg/day, which may be given in a divided dose twice daily. In certain embodiments the dose may particularly be 24 mg/day, which may be given as 12 mg bid, or other doses such as, e.g., 20 mg/day, 16 mg/day, or 12 mg/day, which may be given as 10 mg bid, 8 mg bid, or 6 mg bid, respectively. In other embodiments, the patient may be administered an active metabolite of iloperidone such as P88, i.e. S-P88; R-P88; or a pharmaceutically acceptable salt of any of the foregoing. Additionally, the patient may have a personal history of gout, i.e., may have experienced symptoms of gout previously, or a familial history of gout, for example, a first degree relative such as a sibling or a parent who has a personal history of gout.

According to the present embodiment, the improvement in such treatment includes monitoring the patient's serum urate concentration or level. Such monitoring may include obtaining a biological sample such as, e.g., blood or plasma from the patient; and testing the biological sample to determine the serum urate concentration in the biological sample. The patient's serum urate concentration (SUC) may then be compared to a reference SUC. Reference levels for SUCs are known to those of ordinary skill in the art, but may include, e.g.: 400 μmol/L (male or female); 4.0-8.5 mg/dL or 0.24-0.51 mmol/L (male); about 453 μmol/L (male); 2.7-7.3 mg/dL or 0.16-0.43 mmol/L (female); or about 394 μmol/L (female).

Such monitoring may be performed at one or more milestones relative to treatment of the patient with iloperidone, an active metabolite of iloperidone such as P88, or a pharmaceutically acceptable salt of iloperidone or the metabolite thereof such as, e.g., prior to administration, on or about day 28 of treatment, on or about day 21 of treatment, on or about day 14 of treatment, and on or about day 7 of treatment. In various embodiments, the monitoring may be performed at one, two, three, four, or all five of the foregoing milestones. The monitoring may further be continued after day 28, for example, periodically at intervals of, e.g., every approximately 28 days thereafter. Such periodic monitoring may continue for a prespecified period of time, or throughout a duration of the patient's treatment. Where the periodic monitoring continues for a prespecified period of time, the prespecified period may be tied to a particular unit of time, e.g, as measured in weeks, months, or years. Alternatively, the monitoring may be continued until a milestone that may be defined by the patient reaching a steady state or substantially steady state serum urate concentration, for example, over a certain number of monitoring events.

The method may further include determining whether the patient is currently being treated for gout, e.g., with a uric acid reducing medication such as, e.g., probenecid, allopurinol, febuxostat, or pegloticase.

In the event that the patient's SUC exceeds the applicable reference level, and the patient is not currently being treated for gout, the improved method includes initiating and maintaining treatment with a uric acid reducing medication while the patient is being treated with iloperidone.

In the event that the patient's SUC exceeds the reference level, and the patient is currently being treated for gout, the improved method includes increasing the dose of the uric acid reducing medication being administered to the patient while the patient is being treated with iloperidone. Alternatively and/or additionally, treatment with a different uric acid reducing medication may be initiated, either alone or in combination with the first uric acid reducing medication.

In the event that the patient's SUC is within the reference level, and the patient is not currently being treated for gout, treatment with iloperidone, an active metabolite of iloperidone such as P88, or a pharmaceutically acceptable salt of iloperidone or the metabolite thereof may be maintained in the absence of any uric acid reducing medication, subject to the outcome of any future or ongoing monitoring of the patient's SUC.

In the event that the patient's SUC is within the reference level, and the patient is currently being treated for gout, both the iloperidone (or active metabolite of iloperidone such as P88, or a pharmaceutically acceptable salt of iloperidone or the metabolite thereof) treatment and the uric acid reducing treatment may be maintained at their respective dosages, subject to the outcome of any future or ongoing monitoring of the patient's SUC.

In the event that the patient's SUC is within the reference level, but is trending toward or approaching the reference level, which may occur at, e.g., day 7, day 14, day 21, day 28, or other milestone in treatment, various steps may be taken. In one example, monitoring may be increased in frequency and/or extended in duration. In another example, treatment with a urate-lowering medication or upward dose adjustment of the urate-lowering medication may be employed prophylactically to prevent the patient's SUC from exceeding the reference level. The patient may be deemed to be approaching the reference level when, e.g., the patient's SUC has increased to within about 5%, about 10%, about 15%, or about 20% of the reference level, when the patient's SUC is within a prespecified range encompassing the reference level (e.g., ±5 μmol/L, ±10 μmol/L, ±15 μmol/L, etc.), and so on, as may be understood by one of skill in the art. The patient may also be deemed to be approaching the reference level when, e.g., the patient's SUC has increased over one or more milestones and is on a trajectory that can be extrapolated to a value that exceeds the applicable reference level. For example, if on day 14 or day 21 after initiation of iloperidone treatment, the patient's SUC is on an upward trajectory that has not yet exceeded the reference limit, but it appears that it may or will by day 28 assuming maintenance of treatment, then the patient may be deemed to be approaching the reference level, and prophylactic action may be taken as described herein.

Initiating and/or increasing the dose of a uric acid reducing treatment as described herein provides the benefit of reducing a likelihood that the patient will develop hyperuricemia and suffer negative consequences thereof such as, e.g., an attack of gout.

In various embodiments, the patient may be male, or may be female.

In certain embodiments, the patient may be found or known to be a carrier of a variant SLC2A9 allele that is associated with increases in serum urate concentration that are induced by treatment with iloperidone, an active metabolite of iloperidone such as P88, or a pharmaceutically acceptable salt of iloperidone or the metabolite thereof. For example, the patient may have a genotype that includes one or more SLC2A9 rs7442295 (G) alleles. In certain embodiments, the patient may particularly be homozygous for the rs7442295 (G) allele (rs7442295-G/G) at the SLC2A9 gene. The patient's genotype at the relevant locus may be determined using methods known to one of skill in the art, and/or as described in the Examples set forth herein. In certain embodiments, the patient's SLC2A9 genotype, once known, may contribute to a determination as to whether or how aggressively to treat a patient with a urate-lowering medication according to the improved method described herein. For example, a patient having a homozygous rs7442295-G/G genotype may be treated more aggressively with urate-lowering medication than an individual having a heterozygous rs7442295-A/G genotype or a homozygous rs7442295-A/A genotype.

According to a second embodiment of the disclosure, another improved method may be provided for the treatment of a patient in need thereof with iloperidone, an active metabolite of iloperidone such as P88, or a pharmaceutically acceptable salt of iloperidone or the metabolite thereof, wherein the patient is a carrier of a variant SLC2A9 allele that is associated with increases in serum urate concentration that are induced by, or associated with treatment with iloperidone, an active metabolite of iloperidone such as P88, or a pharmaceutically acceptable salt of iloperidone or the metabolite thereof. The variant SLC2A9 allele may be a rs7442295 (G) allele. In certain embodiments, the patient may carry at least one, or more particularly two rs7442295 (G) alleles, e.g., a rs7442295 (G/G) genotype. The patient's SLC2A9 genotype may be determined as described herein, and/or from consultation with the patient's medical records, in the event that the test, or a test including the relevant locus, had previously been performed. According to the present embodiment, the improved method comprises monitoring a serum urate level of the patient; and in an event that the serum urate level of the patient approaches or exceeds a reference level, initiating treatment with, or increasing a dose of a urate lowering treatment.

The patient may be in need of treatment with iloperidone, an active metabolite of iloperidone such as P88, or a pharmaceutically acceptable salt of iloperidone or the metabolite thereof for control of symptoms and/or prevention of relapse of, e.g., schizophrenia, a schizophreniform disorder, bipolar I disorder, bipolar mania, acute manic and mixed episodes associated with bipolar I disorder, agitation associated with Alzheimer's Disease, agitation associated with dementia, agitation associated with autism, Parkinson's Disease Psychosis, and other psychotic diseases and disorders as is known and understood in the art. In certain embodiments, the patient's treatment may include administration of iloperidone at a dose of up to 24 mg/day, which may be given in a divided dose twice daily. In certain embodiments the dose may particularly be 24 mg/day, which may be given as 12 mg bid, or other doses such as, e.g., 20 mg/day, 16 mg/day, or 12 mg/day, which may be given as 10 mg bid, 8 mg bid, or 6 mg bid, respectively. Additionally, the patient may or may not have a personal history of gout, i.e., may have experienced symptoms of gout previously, or a familial history of gout, such as a first degree relative such as a sibling or a parent who has a personal history of gout.

According to the present embodiment, the improvement in such treatment includes monitoring the patient's serum urate concentration or level. Such monitoring may include obtaining a biological sample such as, e.g., blood or plasma from the patient; and testing the biological sample to determine the serum urate concentration in the biological sample. The patient's serum urate concentration (SUC) may then be compared to a reference SUC. Reference levels for SUCs are known to those of ordinary skill in the art, but may include, e.g.: 400 μmol/L (male or female); 4.0-8.5 mg/dL or 0.24-0.51 mmol/L (male); about 453 μmol/L (male); 2.7-7.3 mg/dL or 0.16-0.43 mmol/L (female); or about 394 μmol/L (female). In certain embodiments, the patient may be male, or may be female.

Monitoring as described herein may be performed at one or more milestones relative to treatment with iloperidone, an active metabolite of iloperidone such as P88, or a pharmaceutically acceptable salt of iloperidone or the metabolite thereof, such as, e.g., prior to administration, on or about day 28 of treatment, on or about day 21 of treatment, on or about day 14 of treatment, and on or about day 7 of treatment. In various embodiments, the monitoring may be performed at one, two, three, four, or all five of the foregoing milestones. Further, the monitoring may be continued after day 28, for example, periodically at intervals of, e.g., every approximately 28 days thereafter. Such periodic monitoring may continue for a prespecified period of time, or throughout a duration of the patient's treatment. Where the periodic monitoring continues for a prespecified period of time, the prespecified period may be tied to a particular unit of time, e.g, as measured in weeks, months, or years. Alternatively, the monitoring may be continued until a milestone that may be defined by the patient reaching a steady state or substantially steady state serum urate concentration, for example, over a certain number of monitoring events.

The method may further include determining whether the patient is currently being treated for gout, e.g., with a uric acid reducing medication such as, e.g., probenecid, allopurinol, febuxostat, or pegloticase, particularly in an embodiment in which the individual is a carrier of a genetic variant that is associated with increases in serum urate concentrations induced by treatment with iloperidone, an active metabolite of iloperidone such as P88, or a pharmaceutically acceptable salt of iloperidone or the metabolite thereof. Such a genotype may be, e.g., rs7442295-G/G.

In the event that the patient's SUC at one or more of day 7, day 14, day 21, day 28, or other milestone exceeds the reference level, and the patient is not currently being treated for gout, the improved method includes initiating treatment with a uric acid reducing medication while the patient is being treated with iloperidone, an active metabolite of iloperidone such as P88, or a pharmaceutically acceptable salt of iloperidone or the metabolite thereof.

In the event that the patient's SUC at one or more of day 7, day 14, day 21, day 28, or other milestone exceeds the reference level, and the patient is currently being treated for gout, the improved method includes increasing the dose of the uric acid reducing medication being administered to the patient while the patient is being treated with iloperidone. Alternatively and/or additionally, treatment with a different uric acid reducing medication may be initiated, either alone or in combination with the first uric acid reducing medication.

In the event that the patient's SUC at day 7, day 14, day 21, day 28, or other milestone is within the reference level, and the patient is not currently being treated for gout, treatment with iloperidone, an active metabolite of iloperidone such as P88, or a pharmaceutically acceptable salt of iloperidone or the metabolite thereof may be maintained in the absence of any uric acid reducing medication, subject to the outcome of any future or ongoing monitoring of the patient's SUC.

In the event that the patient's SUC at day 7, day 14, day 21, day 28, or other milestone is within the reference level, and the patient is currently being treated for gout, both the treatment with iloperidone, an active metabolite of iloperidone such as P88, or a pharmaceutically acceptable salt of iloperidone or the metabolite thereof, and the uric acid reducing medication may be maintained at their respective dosages, subject to the outcome of any future or ongoing monitoring of the patient's SUC.

In the event that the patient's SUC at one or more of day 7, day 14, day 21, day 28, or other milestone is within the reference level, but is trending toward or approaching the reference level, various steps may be taken. In one example, monitoring may be increased in frequency and/or extended in duration. In another example, treatment with a urate-lowering medication or upward dose adjustment of an existing urate-lowering medication may be employed prophylactically to prevent the patient's SUC from exceeding the reference level at a next or future milestone. The patient may be deemed to be approaching the reference level when, e.g., the patient's SUC has increased to within about 5%, about 10%, about 15%, or about 20% of the reference level, when the patient's SUC is within a prespecified range encompassing the reference level (e.g., ±5 μmol/L, ±10 μmol/L, ±15 μmol/L, etc.), and so on, as may be understood by one of skill in the art. The patient may also be deemed to be approaching the reference level when, e.g., the patient's SUC has increased over one or more milestones and is on a trajectory that can be extrapolated to a value that may or will exceed the reference level. For example, if on day 14 or day 21 after initiation of treatment with iloperidone, an active metabolite of iloperidone such as P88, or a pharmaceutically acceptable salt of iloperidone or the metabolite thereof, the patient's SUC is on an upward trajectory that has not yet exceeded the reference limit, but it appears that it may, e.g., by day 28, assuming maintenance of treatment, then the patient may be deemed to be approaching the reference level, and prophylactic action may be taken as described herein.

Initiating and/or increasing the dose of a uric acid reducing medication as described herein provides the benefit of reducing a likelihood that the patient will develop hyperuricemia and suffer negative consequences thereof such as, e.g., an attack of gout.

The skilled artisan will appreciate that additional preferred embodiments may be selected by combining the preferred embodiments above, or by reference to the examples given herein.

### EXAMPLES

### Example 1

A multi-center, randomized, double-blind, placebo-controlled study is performed to evaluate the safety and efficacy of iloperidone for four (4) weeks in the treatment of patients with acute manic episodes associated with bipolar I disorder (“Study 3201”). Patients in the study are randomized to orally receive either iloperidone at a dose of 24 mg/day given in a divided dose as 12 mg twice daily (bid), or matched placebo.

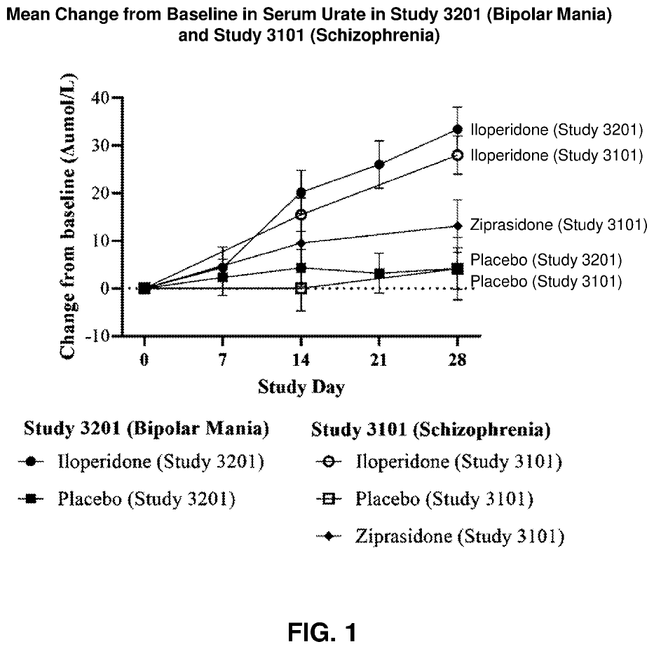

Analysis of laboratory chemistry results reveal that at Day 28, patients receiving iloperidone have 4-fold greater increases in serum urate from baseline compared to placebo groups (33.4 μmol/L change for iloperidone; 4.2 μmol/L change for placebo group). These increases are illustrated in FIG. 1 using solid data points. Patient data are analyzed using an ANCOVA model, and reveal a statistically significant increase in serum urate for iloperidone compared to placebo (Table 1). The LS mean change from baseline to endpoint is an increase of 27.2 μmol/L for iloperidone compared to 0.1 μmol/L for placebo.

TABLE 1
Study 3201 (Bipolar Mania) ANCOVA Results

| | Iloperidone | Placebo | LS Mean | |
|---|---|---|---|---|
| | LS Mean Change | LS Mean Change | Difference | p- |
| Visit | (SE), n | (SE), n | (95% CI) | value |
| Baseline | 323.0 (77.13), 206 | 328.5 (84.6), 208 | — | — |
| Day 7 | −2.7 (−4.07), 199 | −4.0 (−4.11), 196 | 1.3 (−9.29, 11.88) | 0.8098 |
| Day 14 | 12.6 (−4.28), 173 | −2.7 (−4.24), 186 | 15.3 (4.50, 26.15) | 0.0057 |
| Day 21 | 19.1 (−5.24), 157 | −1.9 (−5.07), 166 | 21.0 (8.91, 33.18) | 0.0007 |
| Day 28 | 27.2 (−4.93), 142 | 0.1 (−4.77), 154 | 27.1 (14.94, 39.20) | <0.0001 |

ANCOVA = Analysis of Covariance. CI = Confidence Interval. LS = Least Squares.
LS means, CIs, and p-values are based on ANCOVA model with main effects of treatment group and pooled site, and baseline as a covariant. All p values represent the difference between iloperidone and placebo.

To test the hypothesis of altered urate serum levels in association with variants in the SLC2A9 gene, whole genome sequencing is conducted from blood samples, genotype status is obtained through standard data analysis, and linear models for SLC2A9 variants are performed on urate concentration in samples collected at baseline (corrected for PCs, age, sex) and samples collected at the end of the study (EOS).

One of the most significant variants detected is rs7442295 (p-value 10⁻⁵ (BETA=−23.12). The more common allele, rs7442295 (A), is associated with higher baseline serum urate and hyperuricemia (defined as serum urate concentration greater than 400 μmol/l), with a reported odds ratio of 1.89 (CI: 1.36-2.61, p=5×10⁻⁵). Overall, 79% of white Europeans carry one or two rs7442295 (A) alleles, leading to higher serum urate levels. Each copy leads to an average increase in baseline serum urate concentration of 20 μmol/l, and an approximate doubling of risk for hyperuricemia. The rs7442295 variant has a global minor allele frequency (MAF) of 0.24, with highest MAF reported in patients with African/African American ancestry (0.40).

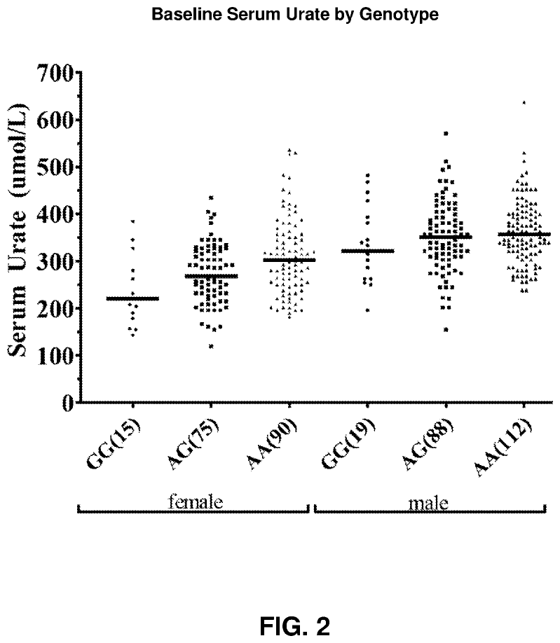

FIG. 2 illustrates baseline evaluations of serum urate according to patient genotype and sex. Data points in the three left-most columns represent female patients (GG(15), AG(75), and AA(90)), while data points in the three right-most columns represent male patients (GG(19), AG(88), and AA(112)). Bars for each column represent the mean level of serum urate. In both males and females, individuals having a genotype of rs7442295 (A/A) have higher mean baseline serum urate concentrations than individuals of the same sex having A/G or G/G genotypes.

Again in both sexes, individuals having a genotype of rs7442295 (G/G) have lower mean baseline serum urate concentrations than individuals of the same sex having A/G or AA genotypes. The minor allele (G) effect at baseline is more pronounced in females, as seen in the larger difference between serum urate levels in GG vs. AA females, as compared to the smaller difference between serum urate levels in GG vs.

AA males. In general, male patients have higher mean serum urate concentrations at baseline than female patients.

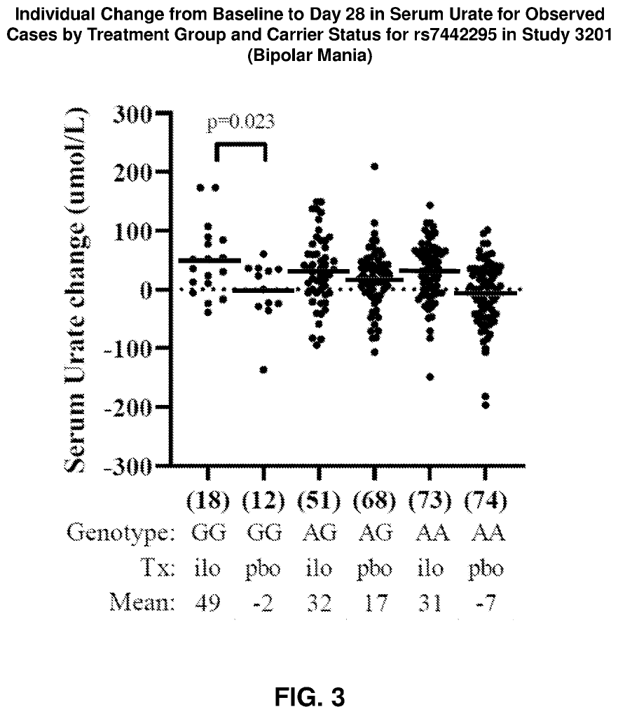

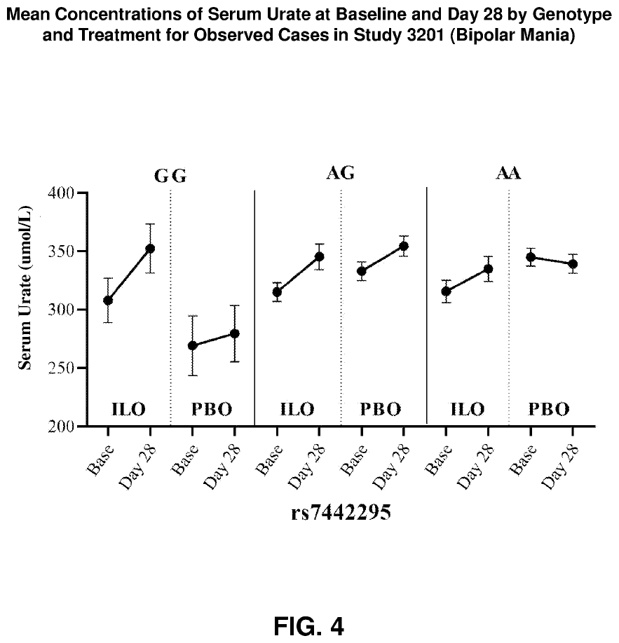

As shown in FIGS. 3-4, the rs7442295 variant of interest has a statistically significant effect on iloperidone-induced change in serum urate levels from baseline to Day 28 in patients with bipolar disorder. FIG. 3 illustrates individual patient changes in serum urate (in μmol/L) from baseline to Day 28, grouped according to treatment group (iloperiodone or placebo) and carrier status for variant rs7442295, while FIG. 4 illustrates mean serum levels for at patient evaluations conducted at baseline and Day 28, separated according to treatment group (iloperidone or placebo) and patient carrier status for rs7442295. The significant interaction affecting change is particularly observed between genotype*sex*treatment (full model: Multiple R-squared: 0.09251, Adjusted R-squared: 0.07078, F-statistic: 4.256 on 8 and 334 DF, p=0.00006942).

This observation is statistically significant when comparing genotypes at BOS as well as when comparing GG with AG and AA genotypes between treatment and placebo group in both males and females combined. Table 2 (below) provides mean change from baseline to Day 28 by treatment group and carrier status for variant rs7442295, and Table 3 (below) provides LS mean change in serum urate from baseline to Day 28 evaluations for serum urate by treatment group and carrier status for variant rs7442295) (SD=standard deviation).

TABLE 2
Mean Change from Baseline to Day 28 in Serum Urate for Observed
Cases by Genotype and Treatment Study 3201 (Bipolar Mania)

| | | Iloperidone | Placebo |
|---|---|---|---|
| Visit | Genotype | Mean Change (SE), n | Mean Change (SE), n |
| Day 28 | AA | 30.5 (50.99), 73 | −6.8 (56.44), 74 |
| Day 28 | AG | 31.9 (59.39), 51 | 17.4 (50.61), 68 |
| Day 28 | GG | 49.1 (60.56), 18 | −2.4 (52.90), 12 |

TABLE 3
LS Mean Change in Serum Urate by Genotype
and Treatment Study 3201 (Bipolar Mania)

| | | Iloperidone | Placebo | LS Mean | |
|---|---|---|---|---|---|
| | | LS Mean Change | LS Mean Change | Difference | |
| Visit | Genotype | (SE), n | (SE), n | (95% CI) | p-value |
| Day | AA | 25.74 (6.53), 73 | −6.17 (6.47), 74 | 31.91 | 0.0003 |
| 28 | | | | (14.82, 49) | |
| Day | AG | 24.62 (7.61), 51 | 9.78 (6.67), 68 | 14.84 | 0.1256 |
| 28 | | | | (−4.18, 33.86) | |
| Day | GG | 40.09 (12.36), 18 | −16.86 (15.37), 12 | 56.95 | 0.0035 |
| 28 | | | | (18.91, 94.99) | |
| EOS | AA | 16.73 (5.66), 101 | −1.5 (5.67), 100 | 18.23 | 0.0176 |
| | | | | (3.19, 33.28) | |
| EOS | AG | 15.05 (6.29), 80 | 6.45 (6.21), 82 | 8.61 | 0.3166 |
| | | | | (−8.27, 25.48) | |
| EOS | GG | 40.4 (12.41), 19 | −15 (14.26), 15 | 55.4 | 0.0032 |
| | | | | (18.68, 92.13) | |

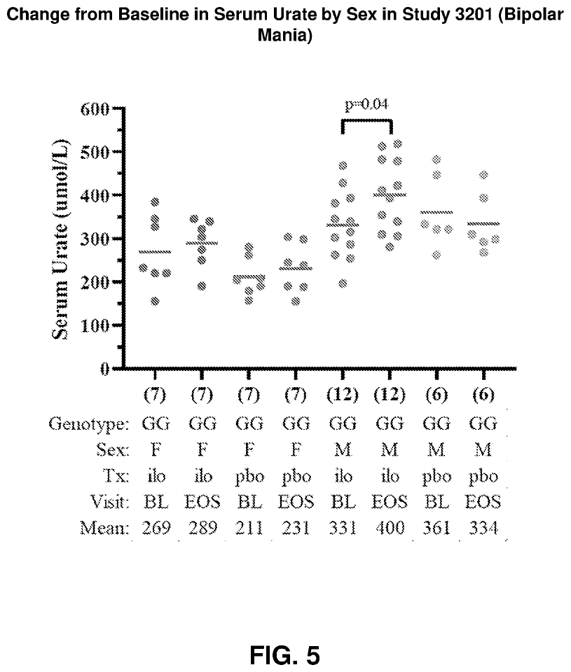

These data show that iloperidone-associated increase in serum urate is significant in individuals who are homozygous for the rs7442295 (G) allele, i.e. have a SLC2A9 rs7442295-G/G genotype. Changes from baseline to study endpoint in serum urate in association with rs7442295-G/G genotype are particularly pronounced in males, as shown in Table 4 and FIG. 5.

TABLE 4
LS Mean Change in Serum Urate by Genotype, Sex, and Treatment in Study 3201 (Bipolar Mania)

| | | | Iloperidone | Placebo | LS Mean | |
|---|---|---|---|---|---|---|
| | | | LS Mean | LS Mean | Difference | p- |
| Visit | Genotype | Sex | Change (SE), n | Change (SE), n | (95% CI) | value |
| Day 28 | AA | F | 25.48 (9.16), 34 | −2.37 (9.06), 34 | 27.85 (2.93, 52.76) | 0.0286 |
| Day 28 | AA | M | 28.89 (8.7), 39 | −6.62 (8.58), 40 | 35.52 (12.82, 58.22) | 0.0023 |
| Day 28 | AG | F | 0.06 (10.14), 27 | 7.96 (9.87), 29 | −7.9 (−34.82, 19.03) | 0.5641 |
| Day 28 | AG | M | 53.41 (10.84), 24 | 12.62 (8.7), 39 | 40.79 (14.61, 66.96) | 0.0024 |
| Day 28 | GG | F | −0.19 (19.49), 7 | −3.27 (20.1), 7 | 3.08 (−50.97, 57.13) | 0.9107 |
| Day 28 | GG | M | 65.61 (15.42), 11 | −37.35 (23.02), 5 | 102.96 (48.93, 156.99) | 0.0002 |
| EOS | AA | F | 14.93 (8.59), 41 | −1.04 (7.81), 48 | 15.97 (−6.71, 38.64) | 0.1669 |
| EOS | AA | M | 20.63 (7.29), 60 | 1.64 (7.87), 52 | 18.99 (−0.89, 38.86) | 0.0611 |
| EOS | AG | F | −2.27 (8.75), 39 | −0.87 (9.26), 36 | −1.4 (−25.85, 23.06) | 0.9106 |
| EOS | AG | M | 32.96 (8.74), 41 | 13.38 (8.26), 46 | 19.58 (−2.91, 42.08) | 0.0878 |
| EOS | GG | F | −6.03 (20.3), 7 | −10.58 (19.66), 8 | 4.54 (−50.43, 59.52) | 0.8709 |
| EOS | GG | M | 67.03 (15.38), 12 | −21.43 (20.27), 7 | 88.45 (38.7, 138.21) | 0.0005 |

Results of ANCOVA model of change from baseline to either day 28 evaluation or end of study evaluation (last observation carried forward).

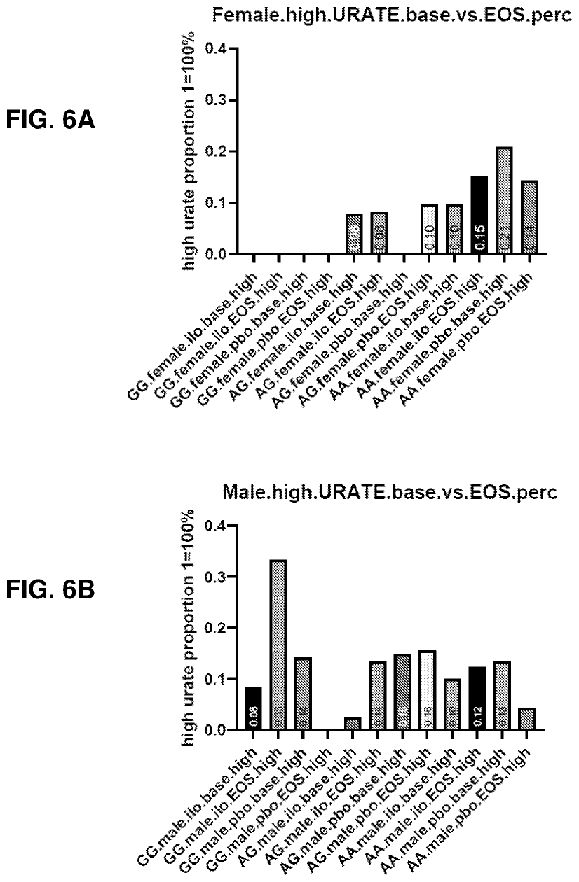

Lab specified ranges for upper level of normal (ULN) values for serum urate measurements are 453 μmol/l for adult male patients and 394 μmol/l for adult female patients. The iloperidone-associated increase in serum urate results in a serum urate concentration that is above ULN value in individuals that are homozygous (GG) for the rs7442295 (G) allele at the SLC2A9 gene, especially in males. As shown in Table 5 (below) and FIGS. 6A-6B, the percentage of male individuals (FIG. 6B) with rs7442295-G/G genotypes and with high urate levels increases by approximately 4-fold upon treatment with iloperidone, from about 8.3% at baseline to about 33.3% at end of study.

TABLE 5
Study 3201: Proportion of Patients with Serum Urate Concentrations
Above the Upper Limit of Normal (ULN) by Sex and SLC2A9
Variant rs7442295 Genotype Subgroups

| | rs7442295 | Male | | Female | |
|---|---|---|---|---|---|
| | genotype | Iloperidone | Placebo | Iloperidone | Placebo |
| Baseline, | GG | 8.3% | 14.3% | 0.0% | 0.0% |
| percentage | | (1/12) | (1/7) | (0/7) | (0/8) |
| of patients | AG | 2.4% | 14.9% | 7.7% | 0.0% |
| above ULN | | (1/41) | (7/47) | (3/39) | (0/36) |
| | AA | 10.0% | 13.5% | 9.5% | 20.1% |
| | | (6/60) | (7/52) | (4/42) | (10/48) |
| EOS, | GG | 33.3% | 0.0% | 0.0% | 0.0% |
| percentage | | (4/12) | (0/6) | (0/7) | (0/70 |
| of patients | AG | 13.5% | 15.5% | 8.1% | 9.7% |
| above ULN | | (5/37) | (7/45) | (3/37) | (3/31) |
| | AA | 12.3% | 4.3% | 15.0% | 14.3% |
| | | (7/57) | (2/46) | (6/40) | (6/42) |

These data show that iloperidone is associated with increases in serum urate compared to placebo. Increases in serum urate levels are nearly twice as large in iloperidone-treated patients homozygous for the common variant rs7442295 (G) in the SLC2A9 transporter gene (i.e., patients with the (GG) genotype).

### Example 2

A study is performed to evaluate the efficacy and safety of iloperidone compared with placebo and active control (ziprasidone) in subjects with acute schizophrenia (“Study 3101”). Patients are randomized to receive one of iloperidone at a dose of 24 mg/day given in a divided dose as 12 mg twice daily (bid), active comparator ziprasidone, or placebo. At Day 28 evaluations, laboratory chemistry analysis shows that patients in the iloperidone treatment arm of the study demonstrate a roughly 4-fold greater increase from baseline in blood levels of uric acid compared to placebo groups, and more than twice the change from baseline observed in the ziprasidone arm. Uric acid results are converted to SI units using the conversion 1 mg/dL=59.5 μmol/L.

Patient data is analyzed using an ANCOVA model, and reveals a statistically significant increase in serum urate for iloperidone compared to placebo groups in patients with acute schizophrenia. The LS mean change from baseline to endpoint shows similar relationships to those described above in Example 1 (see Table 1). Results are provided in Table 6 below, including an increase of 28.0 μmol/L for iloperidone compared to a decrease of 4.2 μmol/L in placebo groups. The ziprasidone treatment arm shows an increase of 13.1 μmol/L, approximately half of that seen in with iloperidone treatment.

TABLE 6
Study 3101 (Schizophrenia) ANCOVA Results

| | Iloperidone | Placebo | LS Mean | |
|---|---|---|---|---|
| | LS Mean Change | LS Mean Change | Difference | |
| Visit | (SE), n | (SE), n | (95% CI) | p-value |
| Day 0 | 318.3 (73.19), 300 | 313.6 (79.73), 174 | — | — |
| Day 14 | 15.5 (58.85), 276 | −29.8 (4.94), 135 | 18.4 (6.55. 29.93) | 0.0024 |
| Day 28 | 28.0 (4.05), 206 | 4.2 (6.07), 98 | 23.8 (9.16. 37.96) | 0.0014 |

ANCOVA = Analysis of Covariance. CI = Confidence Interval. LS = Least Squares.
LS means, CIs, and p-values are based on ANCOVA model with main effects of treatment group and pooled site, and baseline as a covariate. All p values represent the difference between iloperidone and placebo.

Patient genotypes are obtained via a commercial 500k SNP Gene Chip Human Mapping Array. To test the hypothesis of altered urate serum levels in association with variants in SLC2A9, linear models for SLC2A9 variants are performed on urate at baseline levels (corrected for PCs, age, sex) followed by samples collected at end of study (EOS).

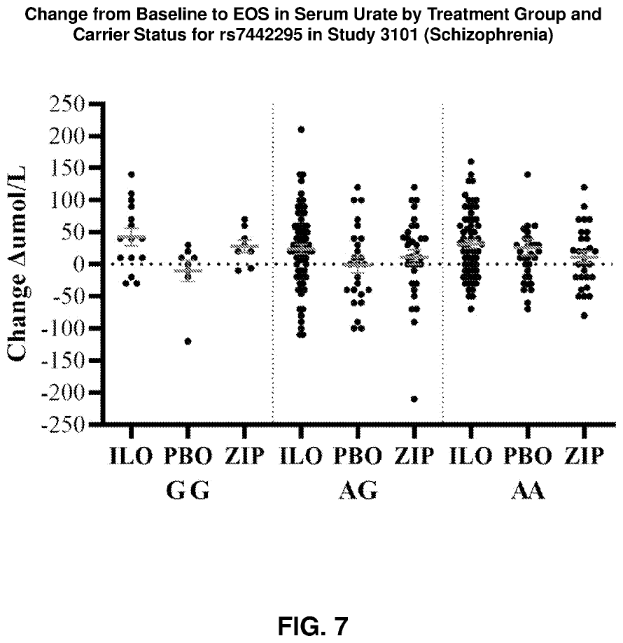

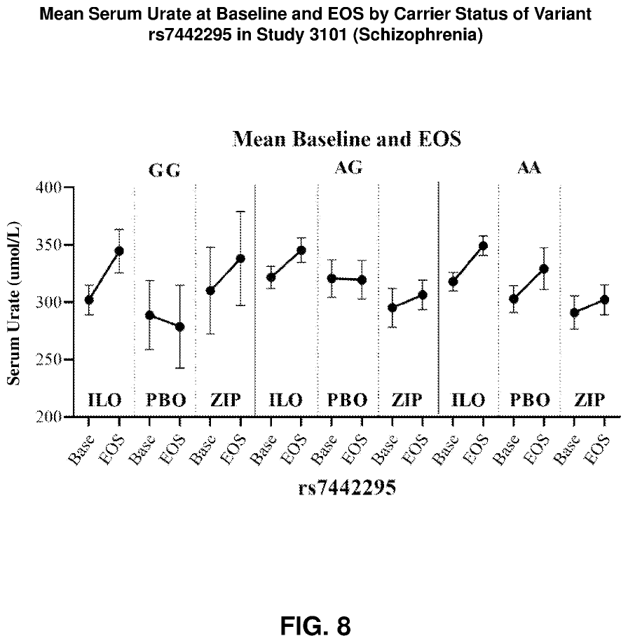

As in Example 1 described herein, the variant of interest, rs7442295 (G) has a statistically significant effect on iloperidone-induced change in serum urate levels from baseline in patients with schizophrenia, as measured at EOS. FIG. 7 illustrates the change from baseline to end of study evaluation for serum urate by treatment group and carrier status for variant rs7442295 in patients with schizophrenia. Individual patient values at baseline and study endpoint are shown for each of the three genotypes (GG, AG, AA) according to treatment arm. FIG. 8 illustrates mean (SEM) serum urate levels at baseline and end of study (EOS) by carrier status of variant rs7442295 in patients with schizophrenia. Results are provided in tabular form in Tables 7-8.

TABLE 7
Mean Change from Baseline to Day 28 in Serum Urate for Observed
Cases by Genotype and Treatment in Study 3101 (Schizophrenia)

| | | Iloperidone | Placebo |
|---|---|---|---|
| Visit | Genotype | Mean Change (SD), n | Mean Change (SD), n |
| Day 28 | AA | 30.5 (54.36), 62 | 26.8 (71.66), 35 |
| Day 28 | AG | 23.3 (61.91), 73 | −1.0 (59.28), 27 |
| Day 28 | GG | 42.7 (52.30), 15 | −10.0 (47.21), 8 |

The observed and derived mean difference is most pronounced in the GG phenotype. This effect is statistically significant according to ANCOVA model analysis (see Table 7).

TABLE 8
LS Mean Change in Serum Urate by Genotype
and Treatment Study 3101 (Schizophrenia)

| | | Iloperidone | Placebo | LS Mean | |
|---|---|---|---|---|---|
| | | LS Mean Change | LS Mean Change | Difference | |
| Visit | Genotype | (SE), n | (SE), n | (95% CI) | p-value |
| Day 28 | AA | 35.5 (7.81), 62 | 24.9 (10.41), 35 | 10.05 (14.90, 35.97) | 0.41 |
| Day 28 | AG | 24.7 (7.27), 73 | −5.5 (12.00), 27 | 30.02 (−2.86, 57.63) | 0.03 |
| Day 28 | GG | 35.9 (15.76), 15 | −16.5 (21.53), 8 | 52.4 (0.33, 104.45) | 0.04 |

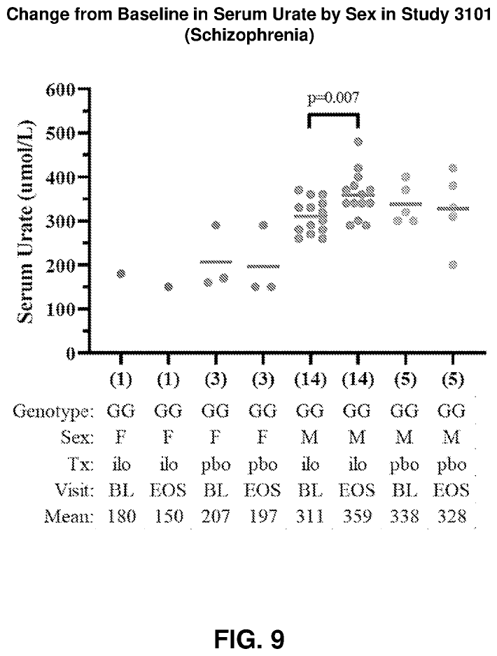

Iloperidone-induced changes from baseline in serum urate in association with GG genotype are particular pronounced in male schizophrenia patients (see FIG. 9). These findings replicate the findings in bipolar disorder discussed above in Example 1.

### CONCLUSIONS

The studies described in Examples 1 and 2 show that iloperidone is associated with increases in serum urate compared to placebo in multiple clinical studies, including in patients with bipolar disorder and those with schizophrenia.

In interaction with iloperidone treatment, the homozygous rs7442295 (G/G) genotype associates with greater iloperidone-induced change in serum urate concentration. Increases in serum urate levels are nearly twice as large in iloperidone-treated patients having a rs7442295 (G/G) genotype in the SLC2A9 transporter gene. This effect is particularly pronounced in male patients.

The replication of these findings across multiple studies and patient populations supports the generalization of these findings to these and other patient populations, including to all individuals who are, will be, or may be treated with iloperidone.

As a result of the interactions described herein, it is recommended that iloperidone-treated patients with a personal or familial history of gout, and carriers of one or more rs7442295 (G) alleles, are monitored for serum urate levels, as such individuals may need to increase the dose of their urate lowering treatments, or commence urate-lowering treatment to prevent or treat gout.

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed, but that the invention will include all embodiments falling within the scope of the appended claims.

What is claimed is:

1. In a method of treatment of a patient in need thereof with a compound that is iloperidone, a pharmaceutically acceptable salt of iloperidone, P88, or a pharmaceutically acceptable salt of P88, wherein the patient has a history of gout, the improvement comprising:
monitoring a serum urate concentration (SUC) of the patient; and
in an event that the SUC approaches or exceeds a reference level, initiating treatment with, or increasing a dose of a urate lowering treatment.

2. The improvement of claim 1, wherein the patient has one or more rs7442295-G alleles at the SLC2A9 gene.

3. The improvement of claim 2, wherein the patient is homozygous for the rs7442295 (G) allele (rs7442295-G/G) at the SLC2A9 gene.

4. In a method of treatment of a patient in need thereof with a compound, wherein the compound is iloperidone, a pharmaceutically acceptable salt of iloperidone, P88, or a pharmaceutically acceptable salt of P88, wherein the patient has a rs7442295 genotype at the SLC2A9 gene that includes one or more rs7442295 (G) alleles, the improvement comprising:
monitoring a serum urate concentration (SUC) of the patient; and
in an event that the SUC of the patient approaches or exceeds a reference level, initiating treatment with, or increasing a dose of a urate lowering treatment.

5. The improvement of claim 1 or claim 4, wherein the compound is iloperidone, and the treatment comprises administering the iloperidone at a dose of up to 24 mg/day.

6. The improvement of claim 5, wherein the dose is 24 mg/day.

7. The improvement of claim 6, wherein the dose of 24 mg/day is given as 12 mg twice daily (bid).

8. The improvement of claim 5, wherein the dose of up to 24 mg/day is given in a divided dose twice daily.

9. The improvement of claim 1 or claim 4, wherein the reference level is 453 μmol/L if the patient is male, and 394 μmol/L if the patient is female.

10. The improvement of claim 1 or claim 4, wherein the patient is female.

11. The improvement of claim 1 or claim 4, wherein the patient is male.

12. The improvement of claim 4, wherein the patient is homozygous for the rs7442295 (G) allele (rs7442295-G/G) at the SLC2A9 gene.

13. The improvement of claim 1 or claim 4, wherein the initiating or increasing reduces a likelihood that the patient will suffer an attack of gout.

14. The improvement of claim 1 or claim 4, wherein the patient is deemed to approach the reference level when the patient has a SUC within about 10% of the reference level.

15. The improvement of claim 1 or claim 4, wherein the monitoring comprises:
obtaining a biological sample from the patient; and
testing the biological sample to determine a concentration of urate in the biological sample.

16. The improvement of claim 15, wherein the monitoring further comprises performing the monitoring at one or more of the following milestones:
on or about day 28 of treatment of the patient with iloperidone,
on or about day 21 of treatment of the patient with iloperidone,
on or about day 14 of treatment of the patient with iloperidone,
on or about day 7 of treatment of the patient with iloperidone, and
prior to treatment of the patient with iloperidone.

17. The improvement of claim 16, wherein the monitoring further comprises performing the monitoring at two or more of the milestones.

18. The improvement of claim 17, wherein the monitoring further comprises performing the monitoring at three or more of the milestones.

19. The improvement of claim 18, wherein the monitoring further comprises performing the monitoring at four or more of the milestones.

20. The improvement of claim 19, wherein the monitoring further comprises performing the monitoring at five of the milestones.

21. The improvement of claim 4, wherein the patient has a history of gout.

22. The improvement of claim 1 or claim 21, wherein the history of gout is a personal history of gout in the patient.

23. The improvement of claim 1 or claim 21, wherein the history of gout is a familial history of gout.

24. The improvement of any one of the preceding claims, wherein the patient is in need of treatment with iloperidone, the pharmaceutically acceptable salt of iloperidone, P88, or the pharmaceutically acceptable salt of P88 for schizophrenia or bipolar disorder.

25. The improvement of any one of the preceding claims, wherein the patient is in need of treatment with iloperidone, the pharmaceutically acceptable salt of iloperidone, P88, or the pharmaceutically acceptable salt of P88 for a schizophreniform disorder, acute manic and mixed episodes associated with bipolar I disorder, agitation associated with Alzheimer's Disease, agitation associated with dementia, agitation associated with autism, Parkinson's Disease Psychosis, or another psychotic disease or disorder.

26. The improvement of any one of the preceding claims, wherein the compound is P88 or a pharmaceutically acceptable salt thereof.
